(19) United States
(12) Patent Application Publication
(10) Pub. No.: US 2024/0133848 A1
(43) Pub. Date: Apr. 25, 2024

(54) INTELLIGENT DETECTION METHOD AND SYSTEM FOR INTERNAL DEFECTS OF WOOD MEMBER WITH A RECTANGULAR SECTION

(71) Applicant: Fuzhou University, Fuzhou (CN)

(72) Inventors: Shaofei Jiang, Fuzhou (CN); Zehui Qiao, Fuzhou (CN); Zhipeng Chen, Fuzhou (CN)

(21) Appl. No.: 18/085,811

(22) Filed: Dec. 21, 2022

(30) Foreign Application Priority Data
Oct. 12, 2022 (CN) 202211244579.7

(51) Int. Cl. G01N 29/44; G01N 29/06; G01N 29/07; G01N 33/46; G06T 7/00

(52) U.S. Cl. CPC G01N 29/4463; G01N 29/069; G01N 29/07; G01N 33/46; G06T 7/0004; G06T 2207/10024; G06T 2207/30161

(57) ABSTRACT

An intelligent detection method and system are provided for identify internal defects of a wood member with a rectangular section. According to the method, collected ultrasonic wave velocity data is corrected to make internal defect characteristics of the rectangular wood member more prominent. In addition, distribution of the corrected ultrasonic wave velocity data in a rectangular cross section of wood member is determined, and gradient visualization processing with red, green and blue (RGB) color is performed according to an ultrasonic wave velocity to obtain a two-dimensional (2D) detection image of a cross section of each layer in the wood member. Then, transformation from each discrete 2D detection plane to a complete three-dimensional (3D) image is performed to precisely detect whether there are defects in the rectangular wood member.

### CROSS REFERENCE TO RELATED APPLICATION

This patent application claims the benefit and priority of Chinese Patent Application No. 202211244579.7, filed on Oct. 12, 2022, the disclosure of which is incorporated by reference herein in its entirety.

### TECHNICAL FIELD

The present disclosure relates to wood member defect detection, and, in particular, to an intelligent detection method and system for internal defects of a wood member with a rectangular section.

### BACKGROUND

Ultrasonic wave is commonly used to detect the internal defects of wood by obtaining the acoustic characteristics of its internal structure. In ultrasonic testing, the propagation velocity of the ultrasonic wave in wood is usually used to reflect the internal defect characteristics of wood.

At present, there are still some deficiencies in the defect detection technology for wood members. The existing wood defect detection technologies are all aimed at logs with an approximate circular section. However, there are also a large quantity of rectangular members in wood structures, such as square columns and rectangular beams. As a result, the detection methods for wood members with circular sections are not completely applicable to wood members with a rectangular section. In addition, due to the anisotropic material properties of wood, the propagation velocity of the ultrasonic wave in the cross section is unevenly distributed, and the ultrasonic wave velocity correction method for the rectangular section is completely different from that of the circular section. Therefore, it would be desirable to develop a defect detection technology for wood members with a rectangular section and provide reliable technical and data support for targeted repair and reinforcement of damaged rectangular wood members.

### SUMMARY

In order to solve the above problems existing in the prior art, the present disclosure provides an intelligent detection method and system for internal defects of a wood member with a rectangular section.

To achieve the above objective, the present disclosure provides the following technical solutions:

An intelligent detection method for internal defects of a wood member with a rectangular section includes the following steps:
obtaining ultrasonic-wave propagation information and the altitude information in the rectangular cross section of the wood member, where the ultrasonic-wave propagation information in the rectangular cross section of the wood member includes: a propagation time and starting and ending coordinates of a propagation path;
determining a propagation distance of each propagation path based on the starting and ending coordinates of the propagation path;
determining ultrasonic wave velocity data in the rectangular cross section of the wood member based on the propagation time and the propagation distance;
obtaining an ultrasonic wave velocity correction coefficient;
correcting the ultrasonic wave velocity data based on the ultrasonic wave velocity correction coefficient to obtain corrected ultrasonic wave velocity data;
determining distribution of the corrected ultrasonic wave velocity data in the rectangular cross section of the wood member, and performing gradient visualization processing with red, green and blue (RGB) color according to an ultrasonic wave velocity to obtain a two-dimensional (2D) detection image of a cross section of each layer in the wood member; obtaining an RGB color threshold of a defect feature and an interlayer interpolation precision;
marking a defect contour in the 2D detection image of the cross section of each layer in the wood member based on the RGB color threshold of the defect feature;
generating an altitude column vector based on the altitude information of the detected rectangular cross section of the wood member;
determining interpolation layer image data between 2D detection images of cross sections of each two layers in the wood member according to the interlayer interpolation precision; and
generating a three-dimensional (3D) detection image based on the 2D detection image of the cross section of each layer in the wood member, the interpolation layer image data, the altitude column vector, and the marked defect contour in the 2D detection image of the cross section of each layer in the wood member.

Preferably, the intelligent detection method further includes the following steps after the step of marking a defect contour in the 2D detection image of the cross section of each layer in the wood member based on the RGB color threshold of the defect feature:
obtaining a quantity of pixels in the image within the defect contour and a quantity of pixels in the 2D detection image; and
according to the quantity of pixels in an image within the defect contour and the quantity of pixels in the 2D detection image, determining a proportion of a defect area in the cross section of each layer in the wood member.

Preferably, the process of correcting the ultrasonic wave velocity data based on the ultrasonic wave velocity correction coefficient to obtain corrected ultrasonic wave velocity data specifically includes:
constructing a circular area by taking a diagonal of the rectangular cross section of the wood member as a diameter;
extending each of the propagation paths in the rectangular cross section of the wood member to intersect the circular area to obtain a chord of the circular area;
determining an included angle between the chord of the circular area and a diameter of the circular area; and
obtaining the corrected ultrasonic wave velocity data based on the ultrasonic wave velocity data along the diagonal propagation path in the wood member section, the ultrasonic wave velocity correction coefficient, and the included angle.

Preferably, the corrected ultrasonic wave velocity data is v:

v=vᵣ+kθ (1),

where θ is the included angle between the chord of the circular area and the diameter of the circular area, vᵣ is the ultrasonic wave velocity data along the diagonal propagation path in the wood member section, and k is the ultrasonic wave velocity correction coefficient.

Preferably, the determining distribution of the corrected ultrasonic wave velocity data in the rectangular cross section of the wood member, and performing gradient visualization processing with RGB color according to an ultrasonic wave velocity to obtain a 2D detection image of a cross section of each layer in the wood member specifically includes:
generating propagation rays based on the starting and ending coordinates of the propagation path;
according to the corrected ultrasonic wave velocity data, performing gradient visualization processing with RGB color on the propagation rays to obtain a ray graph;
after all of the propagation rays in the ray graph are discretized into a quantity of points, iteratively segmenting the propagation rays to obtain segmented rays, where a length of each of the segmented rays is less than or equal to one sixteenth of a shortest propagation ray in the ray graph;
constructing a circular neighborhood by taking each of the segmented rays as a diameter;
determining an ultrasonic wave velocity in each of the segmented rays;
constructing an elliptical neighborhood by taking each of the propagation rays as a major axis and taking one tenth of each of the propagation rays as a minor axis;
after the rectangular cross section of the wood member is discretized into a grid graph, determining grid points in the elliptical neighborhood;
on the basis of the segmented rays, constructing a segmented ray influence area in the elliptical neighborhood;
determining an ultrasonic wave velocity in the segmented ray influence area based on the ultrasonic wave velocity in each of the segmented rays;
determining an ultrasonic wave velocity of the grid points in the elliptical neighborhood based on the ultrasonic wave velocity in the segmented ray influence area;
determining an ultrasonic wave velocity of each grid cell based on the ultrasonic wave velocity of the grid points in the elliptical neighborhood after the rectangular cross section of the wood member is discretized into the grid graph; and
performing gradient visualization processing with RGB color on the ultrasonic wave velocity of each grid cell to obtain the 2D detection image of the cross section of each layer in the wood member.

Preferably, the generating a 3D detection image based on the 2D detection image of the cross section of each layer in the wood member, the interpolation layer image data, the altitude column vector, and the marked defect contour in the 2D detection image of the cross section of each layer in the wood member specifically includes:
converting an RBG value of all of the pixels of the 2D detection image and an RGB interpolation color filling ruler used in the 2D detection image into a hue, saturation, value (HSV) value;
inverting colors of all of the pixels in the 2D detection image of the cross section of each layer into color index values to form a 2D color index matrix;
determining a color index matrix of each interpolation layer between each two layers of 2D detection images; and
based on the 2D color index matrix and the color index matrix of each interpolation layer, transforming the 2D detection image and interpolation layer data of each layer into spatial coordinate information and color information to obtain the 3D detection image.

According to the specific embodiments provided by the present disclosure, the present disclosure discloses the following technical effects:

According to the intelligent detection method for internal defects of the wood member with a rectangular section provided by the present disclosure, the characteristics of internal defects of the wood member is more prominent after correcting the collected ultrasonic wave velocity data. In addition, the distribution of the corrected ultrasonic wave velocity data in the rectangular cross section of the wood member is determined, and the 2D detection image of the cross section of each layer in the wood member is obtained by performing the gradient visualization processing with RGB color according to the ultrasonic wave velocity data. Then, the transformation from each discrete 2D detection plane to the complete 3D image is performed to precisely detect whether there are defects in the rectangular wood member.

Corresponding to the above provided intelligent detection method for internal defects of a wood member with a rectangular section, the present disclosure further provides an intelligent detection system for internal defects of a wood member with a rectangular section, including: a data acquisition module, a data correction module, a detection image generation module, a detection image processing module, a detection image 3D reconstruction module, an analysis server, a display terminal, and a storage server.

The analysis server is connected to the data correction module, the detection image generation module, the detection image processing module, the detection image 3D reconstruction module, the display terminal, and the storage server. The storage server is connected to the data acquisition module, the data correction module, the detection image generation module, the detection image processing module, and the detection image 3D reconstruction module.

The data acquisition module is configured to obtain ultrasonic-wave propagation information and the altitude information in the rectangular cross section of the wood member, and send the ultrasonic-wave propagation information in the rectangular cross section of the wood member and the altitude information of the detected rectangular cross section of the wood member to the data correction module and the storage server. The ultrasonic-wave propagation information in the rectangular cross section of the wood member includes: a propagation time and starting and ending coordinates of a propagation path.

The data correction module is configured to correct ultrasonic wave velocity data based on an ultrasonic wave velocity correction coefficient to obtain corrected ultrasonic wave velocity data.

The detection image generation module is configured to determine distribution of the corrected ultrasonic wave velocity data in the rectangular cross section of the wood member, perform gradient visualization processing with RGB color according to an ultrasonic wave velocity to obtain a 2D detection image of a cross section of each layer in the wood member, and send the 2D detection image to the storage server.

The detection image processing module is configured to extract the 2D detection image stored in the storage server, define an RGB color threshold of a defect feature in the 2D detection image, and send the 2D detection image and the RGB color threshold of the defect feature to the analysis server.

The detection image 3D reconstruction module is configured to generate a 3D detection image based on the 2D detection image of the cross section of each layer in the wood member, interpolation layer image data, an altitude column vector, and a marked defect contour in the 2D detection image of the cross section of each layer in the wood member.

The analysis server is configured to determine a propagation distance of each propagation path based on the starting and ending coordinates of the propagation path, determine the ultrasonic wave velocity data in the rectangular cross section of the wood member based on the propagation time and the propagation distance, mark the defect contour in the 2D detection image of the cross section of each layer in the wood member based on the RGB color threshold of the defect feature, generate the altitude column vector based on the altitude information of the rectangular cross section of the wood member, determine the interpolation layer image data between 2D detection images of cross sections of each two layers in the wood member according to an interlayer interpolation precision, obtain a quantity of pixels in the image within the defect contour and a quantity of pixels in the 2D detection image, and determine a proportion of a defect area in the cross section of each layer in the wood member according to the quantity of pixels in the image within the defect contour and the quantity of pixels in the 2D detection image. The interlayer interpolation precision is stored in the storage server.

The display terminal is configured to receive and display the 2D detection image sent by the analysis server, the 2D detection image with a defect contour mark, the proportion of the defect area, and the complete 3D detection image of the rectangular wood member.

The storage server is configured to receive and store propagation time data of the ultrasonic wave and the altitude information, receive and store the corrected ultrasonic wave velocity data and the starting and ending coordinates of the propagation path, the 2D detection image, the 2D detection image with the defect contour mark, the proportion of the defect area, as well as the 3D detection image and the numerical order. Moreover, the ultrasonic wave velocity correction coefficients of various tree species are stored in the storage server.

Preferably, the data acquisition module includes a plurality of ultrasonic transducers.

### BRIEF DESCRIPTION OF THE DRAWINGS

Further advantages, features and possible applications of the present invention will be apparent from the following detailed description in connection with the drawings. The accompanying drawings, which are incorporated in and constitute a part of this specification, illustrate one of more embodiments of the invention and, together with the general description given above and the detailed description given below, explain the one or more embodiments of the invention.

FIG. 1 is a flow chart of an intelligent detection method for internal defects of a wood member with a rectangular section according to one embodiment of the described invention.

FIG. 2 is a schematic diagram of an included angle between a chord formed by each propagation ray and a diameter of a circle corresponding to the chord provided in one embodiment.

FIG. 3 is a schematic diagram of the segmentation process of the ultrasonic wave propagation ray provided by one embodiment.

FIG. 4 is a schematic diagram of an elliptical neighborhood and a segmented ray influence area provided by one embodiment.

FIG. 5 is a schematic structural diagram of an intelligent detection system for internal defects of a wood member with a rectangular section according to embodiments of the described invention.

FIG. 6 is a schematic diagram of a layout of the ultrasonic transducers array provided by one embodiment.

### DETAILED DESCRIPTION

The technical solutions of the embodiments of the present disclosure are clearly and completely described below with reference to the accompanying drawings. The described embodiments will be understood to be merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide an intelligent detection method and system for internal defects of a wood member with a rectangular section, which can precisely detect whether there are defects in the rectangular wood member.

To make the above-mentioned objective, features, and advantages of the present disclosure clearer and more comprehensible, the present disclosure will be further described in detail below in conjunction with the accompanying drawings and specific implementations.

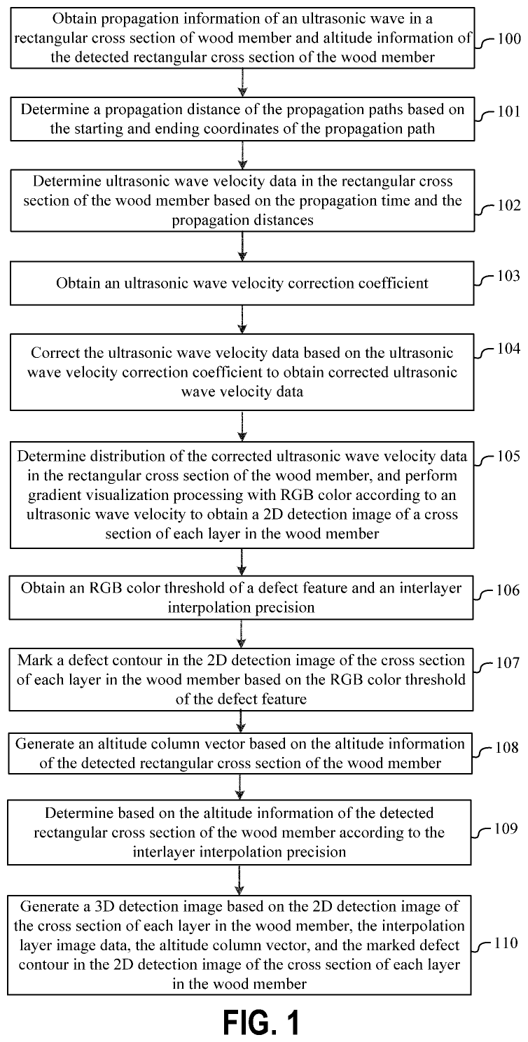

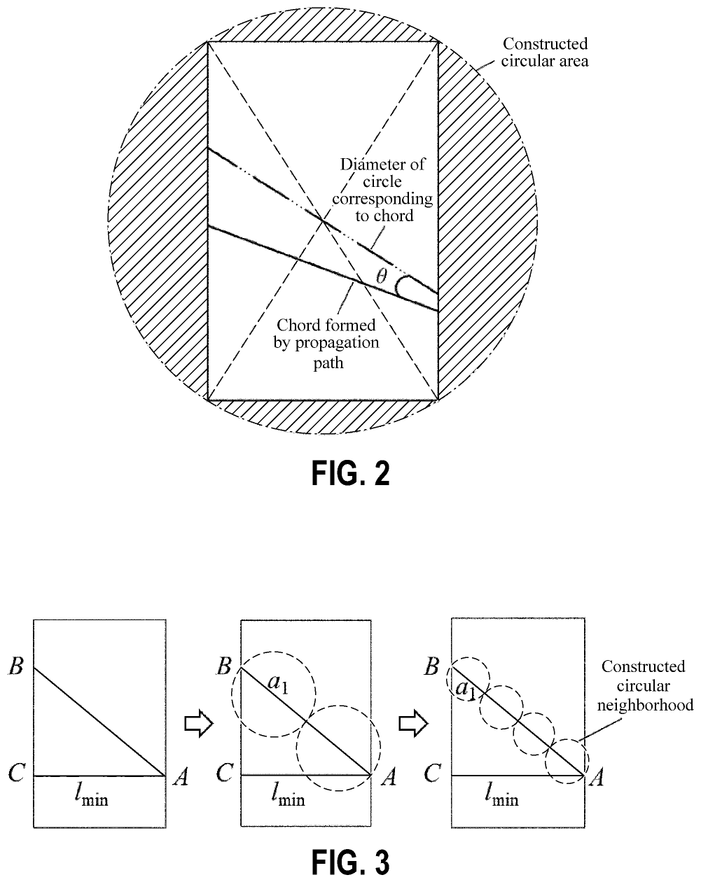

The present disclosure provides an intelligent detection method for internal defects of a wood member with a rectangular section, as shown in FIG. 1, including the following steps.
Step 100: propagation information of an ultrasonic wave in a rectangular cross section of wood member and altitude information of the detected rectangular cross section of the wood member are obtained. The ultrasonic-wave propagation information in the rectangular cross section of the wood member includes: a propagation time and starting and ending coordinates of a propagation path.
Step 101: a propagation distance of the propagation path is determined based on the starting and ending coordinates of the propagation path.
Step 102: ultrasonic wave velocity data in the rectangular cross section of the wood member is determined based on the propagation time and the propagation distance.
Step 103: an ultrasonic wave velocity correction coefficient is obtained.
Step 104: the ultrasonic wave velocity data is corrected based on the ultrasonic wave velocity correction coefficient to obtain corrected ultrasonic wave velocity data. The specific execution process of step 104 is as follows.
   Step 1041: a circular area is constructed by taking a diagonal of the rectangular cross section as a diameter.
   Step 1042: all of the ultrasonic wave propagation rays in the rectangular cross section are extended to intersect the circular area constructed in step 1041, so as to form a chord of the circular area.
   Step 1043: an included angle θ between the chord formed by each propagation ray in step 1042 and a diameter of a circle corresponding to the chord is calculated, as shown in FIG. 2.
   Step 1044: ultrasonic wave velocity correction is performed according to Formula (1) to obtain corrected ultrasonic wave velocity data.

v=vᵣ+kθ (1),

where θ is the included angle between the chord of the circular area and the diameter of the circular area, vᵣ is the ultrasonic wave velocity data along the diagonal propagation path in the wood member section, and k is the ultrasonic wave velocity correction coefficient.
Step 105: distribution of the corrected ultrasonic wave velocity data in the rectangular cross section of the wood member is determined, and gradient visualization processing with RGB color is performed according to an ultrasonic wave velocity to obtain a 2D detection image of a cross section of each layer in the wood member. In the present disclosure, the implementation process of step 105 can include a variety of implementations, for example, one of which is as follows.
   Step 1051: propagation rays are generated based on the starting and ending coordinates of the propagation path.
   Step 1052: according to the corrected ultrasonic wave velocity data, gradient visualization processing with RGB color is performed on the propagation rays to obtain a ray graph.
   Step 1053: after all of the propagation rays in the ray graph are discretized into a quantity of points, the propagation rays are iteratively segmented to obtain segmented rays. A length of each of the segmented rays is less than or equal to one sixteenth of a shortest propagation ray in the ray graph.
   Step 1054: a circular neighborhood is constructed by taking each of the segmented rays as a diameter.
   Step 1055: an ultrasonic wave velocity in each of the segmented rays is determined.
   Step 1056: an elliptical neighborhood is constructed by taking each of the propagation rays as a major axis and taking one tenth of each of the propagation rays as a minor axis.
   Step 1057: after the rectangular cross section of the wood member is discretized into a grid graph, grid points in the elliptical neighborhood are determined.
   Step 1058: on the basis of the segmented rays, a segmented ray influence area is constructed in the elliptical neighborhood.
   Step 1059: an ultrasonic wave velocity in the segmented ray influence area in the elliptical neighborhood is determined based on the ultrasonic wave velocity in each of the segmented rays.
   Step 10511: an ultrasonic wave velocity of the grid points in the elliptical neighborhood is determined based on the ultrasonic wave velocity in the segmented ray influence area.
   Step 10512: an ultrasonic wave velocity of each grid cell after the rectangular cross section of the wood member is discretized into the grid graph is determined based on the ultrasonic wave velocity of the grid points in the elliptical neighborhood.
   Step 10513: gradient visualization processing with RGB color is performed on the ultrasonic wave velocity of each grid cell to obtain the 2D detection image of the cross section of each layer in the wood member.

Another implementation of Step 105 is as follows.
Step 1051: the wave velocity data corrected by Formula (1) and the coordinates of the starting point (xᵢ, yᵢ) and ending point (xⱼ, yⱼ) of each propagation path are input. After propagation rays are generated, all rays are discretized into several points (xₙ^{ij}, yₙ^{ij}) according to Formula (2):

$$\\ \begin{matrix}{x}_{n}^{{}^{} ij} = \{ \begin{matrix}{x}_{i} , & {x}_{i} = {x}_{j} \\ range [ min ( {x}_{i} , {x}_{j} ) , max ( {x}_{i} , {x}_{j} ) ] , & \vert {x}_{i} - {x}_{j} \vert > \vert {y}_{i} - {y}_{j} \vert \\ \frac{{x}_{i} - {x}_{j}}{{y}_{i} - {y}_{j}} \cdot {y}_{n}^{{}^{} ij} + {x}_{i} - \frac{{x}_{i} - {x}_{j}}{{y}_{i} - {y}_{j}} \cdot {y}_{i} , & \vert {x}_{i} - {x}_{j} \vert < \vert {y}_{i} - {y}_{j} \vert\end{matrix} & ( 2 )\end{matrix}$$

$$\\ {y}_{n}^{{}^{} ij} = \{ \begin{matrix}range [ min ( {y}_{i} , {y}_{j} ) , max ( {y}_{i} , {y}_{j} ) ] , & {x}_{i} = {x}_{j} \\ \frac{{y}_{i} - {y}_{j}}{{x}_{i} - {x}_{j}} \cdot {x}_{n}^{{}^{} ij} + {y}_{i} - \frac{{y}_{i} - {y}_{j}}{{x}_{i} - {x}_{j}} \cdot {x}_{i} , & \vert {x}_{i} - {x}_{j} \vert > \vert {y}_{i} - {y}_{j} \vert \\ range [ min ( {y}_{i} , {y}_{j} ) , max ( {y}_{i} , {y}_{j} ) ] , & \vert {x}_{i} - {x}_{j} \vert < \vert {y}_{i} - {y}_{j} \vert\end{matrix} ,$$

where xₙ^{ij} is an abscissa of an n-th discrete point after discretization of a ray formed by an i-th starting point and a j-th ending point, yₙ^{ij} is an ordinate of the n-th discrete point after the discretization of the ray formed by the i-th starting point and the j-th ending point, and the range function represents the value in a certain interval.
Step 1052: after a propagation ray graph is generated, all of the rays are bisected. The circular neighborhood (as shown in FIG. 3) is constructed by taking the length of the segmented ray as the diameter. Formulas (3) to (5) are employed to judge whether the segmented rays pass through the circular neighborhood:

$$\\ \begin{matrix}{L}_{a 1} = \vert \frac{( {y}_{i}^{ap 1} - {y}_{i}^{ap 0} ) \cdot {x}_{n}^{{}^{} ij} + ( {x}_{i}^{ap 0} - {x}_{i}^{ap 1} ) \cdot {y}_{n}^{{}^{} ij} + ( {x}_{i}^{ap 1} \cdot {y}_{i}^{ap 0} ) - ( {x}_{i}^{ap 0} \cdot {y}_{i}^{ap 1} )}{\sqrt{{( {x}_{i}^{ap 0} - {x}_{i}^{ap 1} )}^{2} + {( {y}_{i}^{ap 0} - {y}_{i}^{ap 1} )}^{2}}} \vert , & ( 3 )\end{matrix}$$

$$\\ \begin{matrix}{L}_{b 1} = \vert \frac{( {y}_{i}^{bp 1} - {y}_{i}^{bp 0} ) \cdot {x}_{n}^{{}^{} ij} + ( {x}_{i}^{bp 0} - {x}_{i}^{bp 1} ) \cdot {y}_{n}^{{}^{} ij} + ( {x}_{i}^{bp 1} \cdot {y}_{i}^{bp 0} ) - ( {x}_{i}^{bp 0} \cdot {y}_{i}^{bp 1} )}{\sqrt{{( {x}_{i}^{bp 0} - {x}_{i}^{bp 1} )}^{2} + {( {y}_{i}^{bp 0} - {y}_{i}^{bp 1} )}^{2}}} \vert , and & ( 4 )\end{matrix}$$

$$\\ \begin{matrix}f ( x , y ) = \{ \begin{matrix}1 , & {L}_{{b}_{1}}^{2} / ( {( {x}_{i}^{ap 0} - {x}_{i}^{ap 1} )}^{2} + {( {y}_{i}^{ap 0} - {y}_{i}^{ap 1} )}^{2} ) + {L}_{{a}_{1}}^{2} / ( {( {x}_{i}^{bp 0} - {x}_{i}^{bp 1} )}^{2} + {( {y}_{i}^{bp 0} - {y}_{i}^{bp 1} )}^{2} ) \leq 1 \\ 0 , & {L}_{{b}_{1}}^{2} / ( {( {x}_{i}^{ap 0} - {x}_{i}^{ap 1} )}^{2} + {( {y}_{i}^{ap 0} - {y}_{i}^{ap 1} )}^{2} ) + {L}_{{a}_{1}}^{2} / ( {( {x}_{i}^{bp 0} - {x}_{i}^{bp 1} )}^{2} + {( {y}_{i}^{bp 0} - {y}_{i}^{bp 1} )}^{2} ) > 1\end{matrix} , & ( 5 )\end{matrix}$$

$$\\ \begin{matrix}{v}_{j , m}^{{}^{} ( k + 1 )} = {\sum }_{{i}^{{}^{} ( k + 1 )} = 1}^{{n}^{{}^{} ( k + 1 )}} {v}_{i}^{( k )} / {n}^{{}^{} ( k + 1 )} , & ( 6 )\end{matrix}$$

where xᵢ^{ap0} and xᵢ^{ap1} represent abscissas of the starting point and ending point of the segmented ray in an i-th circular neighborhood respectively, yᵢ^{ap0} and yᵢ^{ap1} represent ordinates of the starting point and ending point of the segmented ray in the i-th circular neighborhood respectively, xᵢ^{bp0} and xᵢ^{bp1} represent abscissas of the starting point and ending point of a diameter perpendicular to the segmented ray in the i-th circular neighborhood respectively, and yᵢ^{bp0} and yᵢ^{bp1} represent ordinates of the starting point and ending point of a diameter perpendicular to the segmented ray in the i-th circular neighborhood respectively. If f(x,y)≤1, it means that the point is in the circular neighborhood, and the segmented ray to which the discrete point belongs is marked.
Step 1053: the wave velocity of segmented ray is estimated according to Formula (6):
   where v_{j,m}^{(k+1)} is an estimated wave velocity of the circular neighborhood corresponding to an m-th segmented ray of a j-th original ray after (k+1)-th segmentation, vᵢ^{(k)} is a velocity of a ray passing through the circular neighborhood during (k+1)-th segmentation after k-th segmentation, and n^{(k+1)} is a total quantity of segmented rays passing through the circular neighborhood during the (k+1)-th segmentation.
Step 1054: steps 1052 and 1053 are repeated until all segmented rays reach the iteration termination condition shown in Formula (7):

lⱼ^{(k)}≤lₘᵢₙ/16 (7),

where i is a length of the j-th ray after k times of segmentation, lₘᵢₙ is a length of a shortest ray among all original rays. The above segmentation process is shown in FIG. 3.
Step 1055: an elliptical neighborhood is constructed by taking the unsegmented original ray as a major axis according to Formula (8).

b₂/a₂=0.1 (8),

where a₂ is the major axis of the ellipse, that is, the unsegmented original ray, and b₂ is the minor axis of the ellipse.

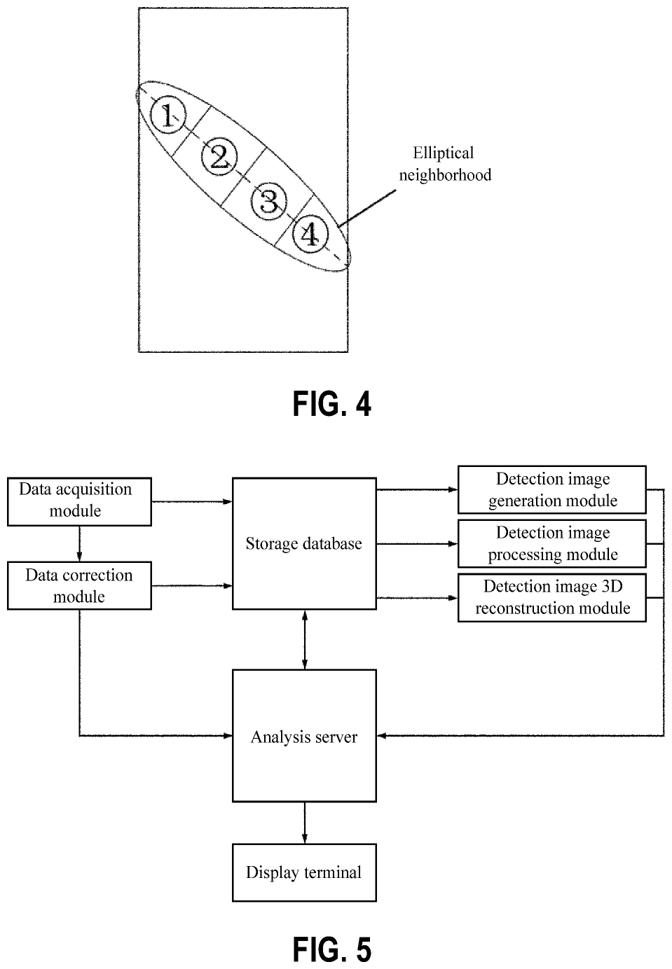

The detected rectangular cross section is discretized into a grid graph, and all grid points in the elliptical neighborhood constructed in step 1055 are recorded according to Formulas (9) to (11):

$$\\ \begin{matrix}{L}_{a 2} = \vert \frac{( {y}_{j}^{ap 1} - {y}_{j}^{ap 0} ) \cdot {x}_{ij}^{{}^{} grid} + ( {x}_{j}^{ap 0} - {x}_{j}^{ap 1} ) \cdot {y}_{ij}^{{}^{} grid} + ( {x}_{j}^{ap 1} \cdot {y}_{j}^{ap 0} ) - ( {x}_{j}^{ap 0} \cdot {y}_{j}^{ap 1} )}{\sqrt{{( {x}_{j}^{ap 0} - {x}_{j}^{ap 1} )}^{2} + {( {y}_{j}^{ap 0} - {y}_{j}^{ap 1} )}^{2}}} \vert , & ( 9 )\end{matrix}$$

$$\\ \begin{matrix}{L}_{b2} = \vert \frac{( {y}_{j}^{bp 1} - {y}_{j}^{bp 0} ) \cdot {x}_{ij}^{{}^{} grid} + ( {x}_{j}^{bp 0} - {x}_{j}^{bp 1} ) \cdot {y}_{ij}^{{}^{} grid} + ( {x}_{j}^{bp 1} \cdot {y}_{j}^{bp 0} ) - ( {x}_{j}^{bp 0} \cdot {y}_{j}^{bp 1} )}{\sqrt{{( {x}_{j}^{bp 0} - {x}_{j}^{bp 1} )}^{2} + {( {y}_{j}^{bp 0} - {y}_{j}^{bp 1} )}^{2}}} \vert , and & ( 10 )\end{matrix}$$

$$\\ \begin{matrix}g ( x , y ) = \{ \begin{matrix}1 , & {L}_{{b}_{2}}^{2} / ( {( {x}_{j}^{ap 0} - {x}_{j}^{ap 1} )}^{2} + {( {y}_{j}^{ap 0} - {y}_{j}^{ap 1} )}^{2} ) + {L}_{{a}_{2}}^{2} / ( {( {x}_{j}^{bp 0} - {x}_{j}^{bp 1} )}^{2} + {( {y}_{j}^{bp 0} - {y}_{j}^{bp 1} )}^{2} ) \leq 1 \\ 0 , & {L}_{{b}_{2}}^{2} / ( {( {x}_{j}^{ap 0} - {x}_{j}^{ap 1} )}^{2} + {( {y}_{j}^{ap 0} - {y}_{j}^{ap 1} )}^{2} ) + {L}_{{a}_{2}}^{2} / ( {( {x}_{j}^{bp 0} - {x}_{j}^{bp 1} )}^{2} + {( {y}_{j}^{bp 0} - {y}_{j}^{bp 1} )}^{2} ) > 1\end{matrix} , & ( 11 )\end{matrix}$$

where xⱼ^{ap0} and xⱼ^{ap1} represent abscissas of the starting point and ending point of the major axis in the j-th elliptical neighborhood respectively, yⱼ^{ap0} and yⱼ^{ap1} represent ordinates of the starting point and ending point of the major axis in the j-th elliptical neighborhood respectively, xⱼ^{bp0} and xⱼ^{bp1} represent abscissas of the starting point and ending point of the minor axis in the j-th elliptical neighborhood respectively, yⱼ^{bp0} and xⱼ^{bp1} represent ordinates of the starting point and ending point of the minor axis in the j-th elliptical neighborhood respectively, and xᵢⱼ^{grid} and yᵢⱼ^{grid} represent the abscissa and ordinate of the grid points respectively. If g(x,y)≤1, the grid is in the elliptical neighborhood.
Step 1056: on the basis of the segmented rays generated in steps 1052 and 1054, a segmented ray influence area is constructed in the elliptical neighborhood constructed in step 1055 according to Formula (12), as shown in FIG. 4:

$$\\ \begin{matrix}h ( x , y ) = \{ \begin{matrix}1 , & {θ}^{′} \leq π / 2 \\ 0 , & {θ}^{′} > π / 2\end{matrix} , & ( 12 )\end{matrix}$$

where θ′ is an included angle between a segmented ray and a straight line connecting any point in the original ray influence area and the ending point of the segmented ray.

The ultrasonic wave velocities of all grids in the rectangular cross section are calculated according to Formula (13), and coloring with RGB colors is performed according to the wave velocity:

$$\\ \begin{matrix}{v}_{xy} = {\sum }_{k = 1}^{N} {v}_{k}^{{}^{} ′} / N , & ( 13 )\end{matrix}$$

where v y is an estimated wave velocity of any grid cell, v′ₖ is a corresponding value of the segmented ellipse affecting the grid cell, and N is a total quantity of segmented ray influence areas simultaneously affecting the grid cell.

The intelligent detection method for internal defects of a wood member with a rectangular section continues with:
Step 106: an RGB color threshold of a defect feature and an interlayer interpolation precision are obtained.
Step 107: a defect contour in the 2D detection image of the cross section of each layer in the wood member is marked based on the RGB color threshold of the defect feature.
Step 108: an altitude column vector is generated based on the altitude information of the detected rectangular cross section of the wood member.
Step 109: interpolation layer image data between 2D detection images of cross sections of each two layers in the wood member is determined according to the interlayer interpolation precision.
Step 110: a 3D detection image is generated based on the 2D detection image of the cross section of each layer in the wood member, the interpolation layer image data, the altitude column vector, and the marked defect contour in the 2D detection image of the cross section of each layer in the wood member. The implementation process of step 110 can be as follows.
   Step 1101: the 2D detection image, the altitude of each detected cross section, and the interlayer interpolation precision are input.
   Step 1102: all pixel points of the 2D detection image and the RGB interpolation color filling ruler used in the detection image are converted from RBG value to HSV value. (x₁ᵢ, y₁ᵢ, z₁ᵢ) represents the HSV value of each pixel of the detection image, and (x₂ⱼ, y₂ⱼ, z₂ⱼ) represents the HSV value of the interpolation color filling ruler.
   Step 1103: the colors of all pixels in the detection image of each layer are inverted into the color index values according to Formula (14), to form a corresponding 2D color index matrix map:

$$\\ \begin{matrix}{index}_{i} = location \{ min [ {x}_{1 i} \cdot {( \begin{matrix}1 \\ 1 \\ … \\ 1\end{matrix} )}_{j \times 1} - ( \begin{matrix}{x}_{21} \\ {x}_{22} \\ … \\ {x}_{2 j}\end{matrix} ) ] \} , & ( 14 )\end{matrix}$$

where indexᵢ represents the color index value of the pixel, x₁ᵢ represents the HSV hue value of the i-th pixel in the detection image, x₂ⱼ represents the HSV hue value of the interpolation color filling ruler, the min function is used to find the minimum element in the column vector, and the location function is used to find the quantity of rows of the minimum element obtained by the min function in the column vector.
Step 1104: a color index matrix of each interpolation layer between two original detection image layers is calculated according to Formulas (15) To (17).

$$\\ \begin{matrix}{ratio\_b}_{m} = \frac{precision \cdot m}{{altitude}_{k + 1} - {altitude}_{k}} , & ( 15 )\end{matrix}$$

$$\\ \begin{matrix}{ratio\_a}_{m} = 1 - ratio\_b , and & ( 16 )\end{matrix}$$

$$\\ \begin{matrix}{map}_{m} = {map}_{k} \cdot ratio\_b + {map}_{k + 1} \cdot ratio\_a , & ( 17 )\end{matrix}$$

where ratio_bₘ represents an interpolation weight of the m-th interpolation layer and the original image of the k-th layer, ratio_aₘ represents an interpolation weight of the interpolation layer and the original image of the (k+1)-th layer, mapₘ represents a color index matrix of the m-th interpolation layer, mapₖ represents a color index matrix of the original image of the k-th layer, mapₖ₊₁ represents a color index matrix of the original image of the (k+1)-th layer, altitudeₖ represents an altitude of the original image of the k-th layer, altitudeₖ₊₁ represents an altitude of the original image of the (k+1)-th layer, and precision represents the interpolation precision.
Step 1105: The original image and interpolation layer data of each layer are transformed into spatial coordinate information and color information.
Step 1106: the defect feature threshold is set to eliminate healthy areas.

In order to precisely visualize the defect location, after step 107 above, the detection method provided by the present disclosure further performs the following steps.

A quantity of pixels in the image within the defect contour and a quantity of pixels in the 2D detection image are obtained.

According to the quantity of pixels in an image within the defect contour and the quantity of pixels in the 2D detection image, a proportion of a defect area in the cross section of each layer in the wood member is determined.

Corresponding to the above provided intelligent detection method for internal defects of a wood member with a rectangular section, the present disclosure further provides an intelligent detection system for internal defects of a wood member with a rectangular section, as shown in FIG. 5, including: a data acquisition module, a data correction module, a detection image generation module, a detection image processing module, a detection image 3D reconstruction module, an analysis server, a display terminal, and a storage server.

The analysis server is connected to the data correction module, the detection image generation module, the detection image processing module, the detection image 3D reconstruction module, the display terminal, and the storage server. The storage server is connected to the data acquisition module, the data correction module, the detection image generation module, the detection image processing module, and the detection image 3D reconstruction module.

The data acquisition module is configured to obtain ultrasonic-wave propagation information and the altitude information in the rectangular cross section of the wood member, and send the ultrasonic-wave propagation information in the rectangular cross section of the wood member and the altitude information of the detected rectangular cross section of the wood member to the data correction module and the storage server. The ultrasonic-wave propagation information in the rectangular cross section of the wood member includes: a propagation time and starting and ending coordinates of a propagation path.

The data correction module is configured to correct ultrasonic wave velocity data based on an ultrasonic wave velocity correction coefficient to obtain corrected ultrasonic wave velocity data.

The detection image generation module is configured to determine distribution of the corrected ultrasonic wave velocity data in the rectangular cross section of the wood member, perform gradient visualization processing with RGB color according to an ultrasonic wave velocity to obtain a 2D detection image of a cross section of each layer in the wood member, and send the 2D detection image to the storage server.

The detection image processing module is configured to extract the 2D detection image stored in the storage server, define an RGB color threshold of a defect feature in the 2D detection image, and send the 2D detection image and the RGB color threshold of the defect feature to the analysis server.

The detection image 3D reconstruction module is configured to generate a 3D detection image based on the 2D detection image of the cross section of each layer in the wood member, interpolation layer image data, an altitude column vector, and a marked defect contour in the 2D detection image of the cross section of each layer in the wood member.

The analysis server is configured to determine a propagation distance of each propagation path based on the starting and ending coordinates of the propagation path, determine the ultrasonic wave velocity data in the rectangular cross section of the wood member based on the propagation time and the propagation distance, mark the defect contour in the 2D detection image of the cross section of each layer in the wood member based on the RGB color threshold of the defect feature, generate the altitude column vector based on the altitude information of the rectangular cross section of the wood member, determine the interpolation layer image data between 2D detection images of cross sections of each two layers in the wood member according to an interlayer interpolation precision, obtain a quantity of pixels in the image within the defect contour and a quantity of pixels in the 2D detection image, and determine a proportion of a defect area in the cross section of each layer in the wood member according to the quantity of pixels in the image within the defect contour and the quantity of pixels in the 2D detection image. The interlayer interpolation precision is stored in the storage server.

The display terminal is configured to receive and display the 2D detection image sent by the analysis server, the 2D detection image with a defect contour mark, the proportion of the defect area, and the complete 3D detection image of the rectangular wood member.

The storage server is configured to receive and store propagation time data of the ultrasonic wave and the altitude information, receive and store the corrected ultrasonic wave velocity data and the starting and ending coordinates of the propagation path, the 2D detection image, the 2D detection image with the defect contour mark, the proportion of the defect area, as well as the 3D detection image and a numerical order, and store ultrasonic wave velocity correction coefficients of various tree species.

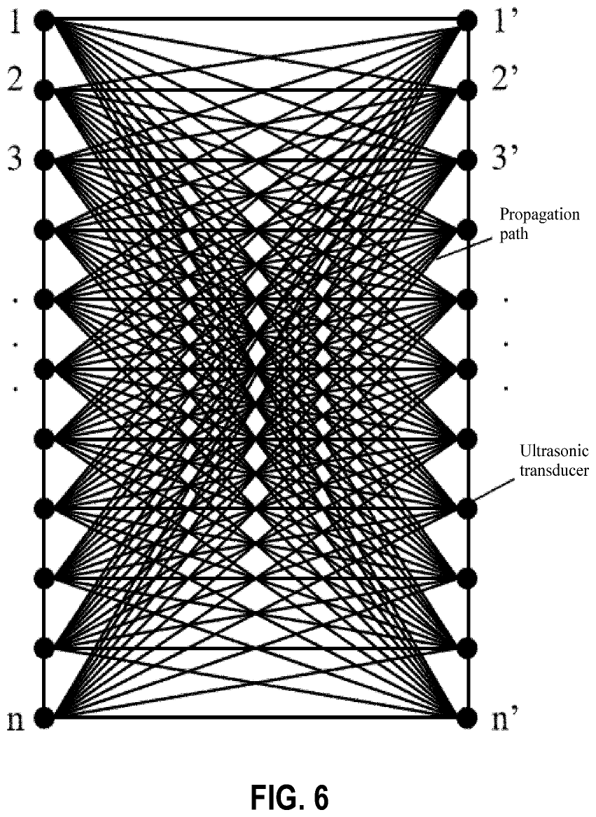

The schematic diagram of the layout of the ultrasonic transducers army is shown in FIG. 6.

Based on the above description, compared with the prior art, the present disclosure also has the following beneficial effects:
1. According to the ultrasonic intelligent detection system for internal defects of the wood member with a rectangular section based on image analysis provided by the present disclosure, the detection image generation module and the detection image 3D reconstruction module visualize the internal defects of the wood member with a rectangular section, making the defect information of 2D cross sections and 3D complete members more intuitive. The defect contour and the proportion of the defect area of the 2D detection image provided by the detection image analysis module provide reliable reference data for evaluating the damage of the wood member with a rectangular section.
2. The ultrasonic wave velocity correction method provided by the present disclosure corrects the collected ultrasonic wave velocity data, making the internal defect characteristics of the rectangular wood member more prominent, which significantly improves the precision of the detection image and makes up for the shortcomings of the existing research on the wave velocity correction method of the rectangular section.
3. The calculation method for detecting the distribution of the ultrasonic wave velocity in the cross section provided by the present disclosure realizes the 2D visual detection of the internal defects of the wood member with a rectangular section.
4. The 3D detection image reconstruction method of the rectangular wood member provided by the present disclosure realizes the transformation from each discrete 2D detection plane to the complete 3D image, and sets the defect feature threshold to eliminate the healthy area, making the internal defect distribution more intuitive.

Each embodiment of the present specification is described in a progressive manner, each embodiment focuses on the difference from other embodiments, and the same and similar parts between the embodiments may refer to each other.

Specific examples are used herein to explain the principles and embodiments of the present disclosure. The foregoing description of the embodiments is merely intended to help understand the method of the present disclosure and its core ideas; besides, various modifications may be made by those of ordinary skill in the art to specific embodiments and the scope of application in accordance with the ideas of the present disclosure. In conclusion, the content of the present specification shall not be construed as limitations to the present disclosure.

The embodiments described above are only descriptions of preferred embodiments of the present invention and are not intended to limit the scope of the present invention. Various variations and modifications can be made to the technical solution of the present invention by those of ordinary skills in the art, without departing from the design and spirit of the present invention. The variations and modifications should all fall within the claimed scope defined by the claims of the present invention.

What is claimed is:

1. An intelligent detection method for internal defects of a wood member with a rectangular section, the method comprising:
obtaining ultrasonic-wave propagation information and an altitude information in the rectangular cross section of the wood member, wherein the ultrasonic-wave propagation information in the rectangular cross section of the wood member comprises: a propagation time and starting and ending coordinates of a propagation path;
determining a propagation distance of each propagation path based on the starting and ending coordinates of the propagation path;
determining an ultrasonic wave velocity data in the rectangular cross section of the wood member based on the propagation time and the propagation distance;
obtaining an ultrasonic wave velocity correction coefficient;
correcting the ultrasonic wave velocity data based on the ultrasonic wave velocity correction coefficient to obtain corrected ultrasonic wave velocity data;
determining distribution of the corrected ultrasonic wave velocity data in the rectangular cross section of the wood member, and performing gradient visualization processing with red, green and blue (RGB) color according to an ultrasonic wave velocity to obtain a two-dimensional (2D) detection image of a cross section of each layer in the wood member;
obtaining a RGB color threshold of a defect feature and an interlayer interpolation precision;
marking a defect contour in the 2D detection image of the cross section of each layer in the wood member based on the RGB color threshold of the defect feature;
generating an altitude column vector based on the altitude information of the detected rectangular cross section of the wood member;
determining an interpolation layer image data between 2D detection images of cross sections of each two layers in the wood member according to the interlayer interpolation precision; and
generating a three-dimensional (3D) detection image based on the 2D detection image of the cross section of each layer in the wood member, the interpolation layer image data, the altitude column vector, and the marked defect contour in the 2D detection image of the cross section of each layer in the wood member.

2. The intelligent detection method for internal defects of a wood member with a rectangular section of claim 1, further comprising, after the step of marking a defect contour in the 2D detection image of the cross section of each layer in the wood member based on the RGB color threshold of the defect feature:
obtaining a quantity of pixels in the image within the defect contour and a quantity of pixels in the 2D detection image; and
according to the quantity of pixels in an image within the defect contour and the quantity of pixels in the 2D detection image, determining a proportion of a defect area in the cross section of each layer in the wood member.

3. The intelligent detection method for internal defects of a wood member with a rectangular section of claim 1, wherein the step of correcting the ultrasonic wave velocity data based on the ultrasonic wave velocity correction coefficient to obtain corrected ultrasonic wave velocity data specifically comprises:
constructing a circular area by taking a diagonal of the rectangular cross section of the wood member as a diameter;
extending each of the propagation paths in the rectangular cross section of the wood member to intersect the circular area to obtain a chord of the circular area;
determining an included angle between the chord of the circular area and a diameter of the circular area; and
obtaining the corrected ultrasonic wave velocity data based on the ultrasonic wave velocity data along the diagonal propagation path in the wood member, the ultrasonic wave velocity correction coefficient, and the included angle.

4. The intelligent detection method for internal defects of a wood member with a rectangular section of claim 3, wherein the corrected ultrasonic wave velocity data is v:
v=vᵣ+kθ (1),
wherein θ is the included angle between the chord of the circular area and the diameter of the circular area, vᵣ is the ultrasonic wave velocity data along the diagonal propagation path in the wood member, and k is the ultrasonic wave velocity correction coefficient.

5. The intelligent detection method for internal defects of a wood member with a rectangular section of claim 1, wherein the step of determining distribution of the corrected ultrasonic wave velocity data in the rectangular cross section of the wood member, and performing gradient visualization processing with RGB color according to an ultrasonic wave velocity to obtain a 2D detection image of a cross section of each layer in the wood member specifically comprises:
generating propagation rays based on the starting and ending coordinates of the propagation path;
according to the corrected ultrasonic wave velocity data, performing gradient visualization processing with RGB color on the propagation rays to obtain a ray graph;
after all of the propagation rays in the ray graph are discretized into a quantity of points, iteratively segmenting the propagation rays to obtain segmented rays, wherein a length of each of the segmented rays is less than or equal to one sixteenth of a shortest propagation ray in the ray graph;
constructing a circular neighborhood by taking each of the segmented rays as a diameter;
determining an ultrasonic wave velocity in each of the segmented rays;
constructing an elliptical neighborhood by taking each of the propagation rays as a major axis and taking one tenth of each of the propagation rays as a minor axis;
after the rectangular cross section of the wood member is discretized into a grid graph, determining grid points in the elliptical neighborhood;
on the basis of the segmented rays, constructing a segmented ray influence area in the elliptical neighborhood;
determining an ultrasonic wave velocity in the segmented ray influence area based on the ultrasonic wave velocity in each of the segmented rays;
determining an ultrasonic wave velocity of the grid points in the elliptical neighborhood based on the ultrasonic wave velocity in the segmented ray influence area;
determining an ultrasonic wave velocity of each grid cell based on the ultrasonic wave velocity of the grid points in the elliptical neighborhood after the rectangular cross section of the wood member is discretized into the grid graph; and
performing gradient visualization processing with RGB color on the ultrasonic wave velocity of each grid cell to obtain the 2D detection image of the cross section of each layer in the wood member.

6. The intelligent detection method for internal defects of a wood member with a rectangular section of claim 1, wherein the step of generating a 3D detection image based on the 2D detection image of the cross section of each layer in the wood member, the interpolation layer image data, the altitude column vector, and the marked defect contour in the 2D detection image of the cross section of each layer in the wood member specifically comprises:
converting an RBG value of all of the pixels of the 2D detection image and an RGB interpolation color filling ruler used in the 2D detection image into a hue, saturation, value (HSV) value;
inverting colors of all of the pixels in the 2D detection image of the cross section of each layer into color index values to form a 2D color index matrix;
determining a color index matrix of each interpolation layer between each two layers of 2D detection images; and
based on the 2D color index matrix and the color index matrix of each interpolation layer, transforming the 2D detection image and interpolation layer data of each layer into spatial coordinate information and color information to obtain the 3D detection image.

7. An intelligent detection system for internal defects of a wood member with a rectangular section, comprising:
a data acquisition module,
a data correction module,
a detection image generation module,
a detection image processing module,
a detection image 3D reconstruction module,
an analysis server,
a display terminal, and
a storage server, wherein:
the analysis server is connected to the data correction module, the detection image generation module, the detection image processing module, the detection image 3D reconstruction module, the display terminal, and the storage server; and the storage server is connected to the data acquisition module, the data correction module, the detection image generation module, the detection image processing module, and the detection image 3D reconstruction module;
the data acquisition module is configured to obtain ultrasonic-wave propagation information and an altitude information in the rectangular cross section of the wood member, and send the ultrasonic-wave propagation information in the rectangular cross section of the wood member and the altitude information of the detected rectangular cross section of the wood member to the data correction module and the storage server, wherein the ultrasonic-wave propagation information in the rectangular cross section of the wood member comprises: a propagation time and starting and ending coordinates of a propagation path;
the data correction module is configured to correct ultrasonic wave velocity data based on an ultrasonic wave velocity correction coefficient to obtain corrected ultrasonic wave velocity data;
the detection image generation module is configured to determine distribution of the corrected ultrasonic wave velocity data in the rectangular cross section of the wood member, perform gradient visualization processing with RGB color according to an ultrasonic wave velocity to obtain a 2D detection image of a cross section of each layer in the wood member, and send the 2D detection image to the storage server;
the detection image processing module is configured to extract the 2D detection image stored in the storage server, define an RGB color threshold of a defect feature in the 2D detection image, and send the 2D detection image and the RGB color threshold of the defect feature to the analysis server;
the detection image 3D reconstruction module is configured to generate a 3D detection image based on the 2D detection image of the cross section of each layer in the wood member, interpolation layer image data, an altitude column vector, and a marked defect contour in the 2D detection image of the cross section of each layer in the wood member;
the analysis server is configured to determine a propagation distance of each propagation path based on the starting and ending coordinates of the propagation path, determine the ultrasonic wave velocity data in the rectangular cross section of the wood member based on the propagation time and the propagation distance, mark the defect contour in the 2D detection image of the cross section of each layer in the wood member based on the RGB color threshold of the defect feature, generate the altitude column vector based on the altitude information of the rectangular cross section of the wood member, determine the interpolation layer image data between 2D detection images of cross sections of each two layers in the wood member according to an interlayer interpolation precision, obtain a quantity of pixels in the image within the defect contour and a quantity of pixels in the 2D detection image, and determine a proportion of a defect area in the cross section of each layer in the wood member according to the quantity of pixels in the image within the defect contour and the quantity of pixels in the 2D detection image, wherein the interlayer interpolation precision is stored in the storage server;
the display terminal is configured to receive and display the 2D detection image sent by the analysis server, the 2D detection image with a defect contour mark, the proportion of the defect area, and a complete 3D detection image of the rectangular wood member; and
the storage server is configured to receive and store propagation time data of the ultrasonic wave and the altitude information, receive and store the corrected ultrasonic wave velocity data and the starting and ending coordinates of the propagation path, the 2D detection image, the 2D detection image with the defect contour mark, the proportion of the defect area, as well as the 3D detection image and a numerical order, and store ultrasonic wave velocity correction coefficients of various tree species.

8. The intelligent detection system for internal defects of a wood member with a rectangular section of claim 7, wherein the data acquisition module comprises:
a plurality of ultrasonic transducers.
